(19) United States
(12) Patent Application Publication
(10) Pub. No.: US 2025/0235298 A1
(43) Pub. Date: Jul. 24, 2025

(54) DATA PROCESSING DEVICE AND DATA PROCESSING METHOD

(71) Applicant: MEDIT CORP, Seoul (KR)

(72) Inventor: Sang Hoon KIM, Seoul (KR)

(73) Assignee: MEDIT CORP, Seoul (KR)

(21) Appl. No.: 18/702,131

(22) PCT Filed: Oct. 18, 2022
(86) PCT No.: PCT/KR2022/015866

§ 371 Date: Apr. 17, 2024

(30) Foreign Application Priority Data
Oct. 18, 2021 (KR) 10-2021-0138605
Oct. 17, 2022 (KR) 10-2022-0133575

(51) Int. Cl. A61C 13/34; A61C 7/00

(52) U.S. Cl. CPC A61C 13/34; A61C 7/002

(57) ABSTRACT

Provided is a data processing method including identifying a boundary line selected for generation of a die, from three-dimensional (3D) scan data of an object, and designing a die of the object by connecting the boundary line and a target plane included in a bottom surface of a base model.

### TECHNICAL FIELD

A disclosed embodiment relates to a data processing device and a data processing method, and more particularly, to a device and method for processing an oral image.

### BACKGROUND ART

For the purpose of orthodontic or prosthetic treatment, a dental model may be used. Users such as a dentist may show patients the appearance of teeth before, during, and after orthodontic treatment by using a dental model. In addition, users may observe or manufacture, by using tooth models, parts that are difficult to observe directly or a prosthesis that is difficult to manufacture directly due to the limited space in the mouth.

Dies may be used among tooth models according to a treatment method. In order to secure a space to be covered with a prosthesis such as a crown or a laminate, users may trim a tooth to manufacture a preparation tooth, and restore a manufactured prosthesis to the preparation tooth. Users may check whether a prosthesis fits well with a die having a preparation tooth shape by using the die having the preparation tooth shape. In addition, users may use the die having the preparation tooth shape and a die having an adjacent tooth shape together to check whether there is a collision region between the preparation tooth and the adjacent teeth while the prosthesis is restored. When there is an unnecessary region between the preparation tooth and the adjacent teeth, users may additionally process the prosthesis.

In the related art, dies have been generated using a plaster model. Users have generated a die for an individual tooth by vertically cutting a plaster model of an oral cavity. However, a job of generating a die from a plaster not only takes a lot of time, but is also inconvenient for both users and patients.

### DISCLOSURE

### Technical Solution

A data processing method according to an embodiment may include identifying a boundary line selected for generation of a die from three-dimensional (3D) scan data of an object, and designing a die of the object by connecting the boundary line and a target plane included in a bottom surface of a base model.

### DESCRIPTION OF DRAWINGS

FIG. 1 is a diagram illustrating an oral image processing system according to an embodiment.

FIG. 2 is a diagram illustrating that a data processing device designs a die by using three-dimensional (3D) scan data, according to an embodiment.

FIG. 3 is an internal block diagram of a data processing device according to an embodiment.

FIG. 4 is an internal block diagram of a data processing device according to an embodiment.

FIG. 5A is a diagram illustrating a data processing device designing a die of an object, according to an embodiment, showing the data processing device outputting 3D scan data on a screen.

FIG. 5B is a diagram illustrating a data processing device designing a die of an object, according to an embodiment, showing 3D scan data of a base model including the object viewed from the side.

FIG. 5C is a diagram illustrating a data processing device designing a die of an object, according to an embodiment, showing 3D scan data of a base model viewed from below.

FIG. 6A is a diagram illustrating a case wherein an interference region is included when a data processing device designs a die of an object, according to an embodiment, showing 3D scan data of a top-down view of a base model.

FIG. 6B is a diagram illustrating a case where an interference region is included when a data processing device designs a die of an object, according to an embodiment, showing 3D scan data of the same base model as the base model shown in FIG. 6A viewed from the side.

FIG. 6C is a diagram illustrating a case where an interference region is included when a data processing device designs a die of an object, according to an embodiment, illustrating a case where the same target curved surface as a target curved surface shown in FIGS. 6A and 6B is enlarged and viewed from a different angle.

FIG. 6D is a diagram illustrating a case where an interference region is included when a data processing device designs a die of an object, according to an embodiment, illustrating a case where the same target curved surface as a target curved surface shown in FIGS. 6A and 6B is enlarged and viewed from a different angle.

FIG. 7A is a diagram illustrating a data processing device generating a die by removing an interference region, according to an embodiment, illustrating a case where a boundary line is indicated on the die.

FIG. 7B is a diagram illustrating a data processing device generating a die by removing an interference region, according to an embodiment, illustrating a case where a boundary line is not indicated on the die.

FIG. 8 is a diagram illustrating a data processing device identifying whether there is an interference region when generating a die, according to an embodiment.

FIG. 9 is a diagram illustrating a data processing device removing an interference region, according to an embodiment.

FIG. 10A is a diagram illustrating a data processing device designing a die of an object, according to an embodiment, illustrating the data processing device simultaneously forming N partial layers having a length of an average side of a mesh between a farthest point of a boundary line from a target plane and a closest point of a boundary line from the target plane.

FIG. 10B is a diagram illustrating a data processing device designing a die of an object according to an embodiment, illustrating the data processing device designing the die by filling between a temporary plane and a target plane with M partial layers.

FIG. 11A is a diagram illustrating a data processing device sequentially forming partial layers when forming a die of an object, according to an embodiment.

FIG. 11B is a diagram illustrating a data processing device sequentially forming partial layers when forming a die of an object, according to an embodiment.

FIG. 11C is a diagram illustrating a data processing device sequentially forming partial layers when forming a die of an object, according to an embodiment.

FIG. 11D is a diagram illustrating a data processing device sequentially forming partial layers when forming a die of an object, according to an embodiment.

FIG. 12 is a diagram illustrating a data processing device designing a die of an object, according to an embodiment.

FIG. 13 is a flowchart illustrating a data processing method according to an embodiment.

FIG. 14 is a flowchart illustrating a data processing method according to an embodiment.

FIG. 15 is a flowchart illustrating a data processing method according to an embodiment.

### MODE FOR INVENTION

In an embodiment, the designing of the die of the object may include identifying a projection direction, identifying, as the target plane, a region where a projection line projected in the projection direction from a point of the boundary line meets the bottom surface of the base model, and designing the die of the object by using a mold surrounded by the projection line.

In an embodiment, the data processing method may further include displaying the designed die, and a cross-section of the displayed die may include one single closed curve.

In an embodiment, the designing of the die of the object may include, based on a fact that two or more single closed curves are included in a projection diagram formed by the projection line on a plane of projection, designing the die of the object by using a projection line projected from only points of a boundary line corresponding to a longest single closed curve among the two or more single closed curves.

In an embodiment, the designing of the die of the object may further include generating a mesh by connecting a point of a boundary line corresponding to a remaining single closed curve excluding the longest single closed curve to a point on a projection line at a shortest distance from the point of the boundary line corresponding to the remaining single closed curve, and filling, with the mesh, empty space below the boundary line corresponding to the remaining single closed curve.

In an embodiment, the designing of the die of the object may include obtaining a length of an average side of meshes included in a curved surface surrounded by the boundary line, obtaining a number N of partial layers (N is a natural number) by dividing a distance between a point of the boundary line farthest from the target plane and a point of the boundary line closest to the target plane by the length of the average side of meshes, and designing the die by forming the N partial layers each having the length of the average side of meshes between the farthest point of the boundary line and the closest point of the boundary line.

In an embodiment, the designing of the die by forming the N partial layers may include forming a first partial layer below the farthest point of the boundary line with a projection line projected from the farthest point of the boundary line toward the target plane and having the length of the average side of meshes, obtaining a new boundary line by connecting a line of the first partial layer to a boundary line below which the first partial layer is not formed, and forming a second partial layer below the first partial layer with a projection line projected from a point of the new boundary line toward the target plane and having the length of the average side of meshes.

In an embodiment, the forming of the second partial layer may include, based on a fact that two or more single closed curves are included in a projection diagram formed on the target plane by the projection line projected from the point of the new boundary line, forming the second partial layer by using a projection line projected from only points of the new boundary line corresponding to a longest single closed curve among the two or more single closed curves.

In an embodiment, the designing of the die of the object may include obtaining, as a first plane of projection, a plane that has a slope perpendicular to a normal vector of a closed curve surface generated using points included in the boundary line and is separated by a shortest distance from the boundary line, obtaining a first projection line projected from a point of the boundary line and vertically meeting the first plane of projection, obtaining a second projection line that is projected from a point where the first projection line and the first plane of projection meet and meets a second plane of projection, by using the target plane as the second plane of projection, and designing the die by using a mold surrounded by the first projection line and the second projection line.

In an embodiment, the obtaining of the first projection line may include, based on a fact that two or more single closed curves are included in a projection diagram formed by the first projection line on the first plane of projection, obtaining, as the first projection line, a projection line projected from only points of the boundary line corresponding to a longest single closed curve among the two or more single closed curves.

In an embodiment, the designing of the die of the object may further include generating a mesh by connecting a point of a boundary line corresponding to a remaining single closed curve excluding the longest single closed curve to a point on a projection line at a shortest distance from the point of the boundary line corresponding to the remaining single closed curve, and filling, with the mesh, empty space below the boundary line corresponding to the remaining single closed curve.

In an embodiment, the obtaining of, as the first plane of projection, the plane may include obtaining the closed curved surface generated using the points included in the boundary line, obtaining a direction of the normal vector of the closed curved surface, and obtaining, as the first plane of projection, the plane that has the slope perpendicular to the normal vector and is separated by the shortest distance from the boundary line among planes in contact with or spaced apart from the boundary line.

In an embodiment, the direction of the normal vector may be a direction of a weighted normal vector obtained by adding a weight according to an area of a mesh forming the closed curved surface.

In an embodiment, the designing of the die of the object may include obtaining a number N of partial layers (N is a natural number) by dividing a distance between the farthest point of the boundary line from the first plane of projection and the first plane of projection by the length of the average side of meshes, and forming the N partial layers each having the length of the average side of meshes between the farthest point of the boundary line and the first plane of projection.

In an embodiment, the forming of the N partial layers may include forming a first partial layer below the farthest point of the boundary line with a projection line projected from the farthest point of the boundary line toward the first plane of projection and having the length of the average side of meshes, obtaining a new boundary line by connecting a line of the first partial layer and a boundary line below which the first partial layer is not formed, and forming a second partial layer below the first partial layer with a projection line projected from a point of the new boundary line toward the first plane of projection and having the length of the average side of meshes.

In an embodiment, the forming of the second partial layer may include, based on a fact that two or more single closed curves are included in a projection diagram formed on the first plane of projection by the projection line projected from the point of the new boundary line, forming the second partial layer by using a projection line projected from only points of the new boundary line corresponding to a longest single closed curve among the two or more single closed curves.

In an embodiment, the designing of the die of the object may further include obtaining a number M of partial layers (M is a natural number) by dividing a maximum distance between the first plane of projection and a second plane of projection by the length of the average side of meshes, by using the target plane as the second plane of projection, and forming M or fewer partial layers each having the length of the average side of meshes between the first plane of projection and the second plane of projection.

A data processing device according to an embodiment includes one or more processors configured to execute one or more instructions, wherein the one or more processors execute the one or more instructions to identify a boundary line selected for generation of a die from 3D design a die of the object by connecting the boundary line and a target plane included in a bottom surface of a base model.

In an embodiment, the data processing device may further include a display, the one or more processors may execute the one or more instructions to output the designed die through the display, and a cross-section of the displayed die may include one single closed curve.

A computer-readable recording medium according to an embodiment has recorded thereon a program for implementing a data processing method including identifying a boundary line selected for generation of a die from 3D scan data of an object, and designing a die of the object by connecting the boundary line and a target plane included in a bottom surface of a base model.

The present specification describes the principles of the present application and discloses embodiments such that the scope of right of the present application is clarified and one of ordinary skill in the art may practice the present application. The embodiments may be implemented in various forms.

Throughout the specification, like reference numerals denote like elements. The present specification does not describe all elements of the embodiments, and generic content in the technical field of the present application or redundant content of the embodiments is omitted. The term “part” or “portion” used in the specification may be implemented by software or hardware, and according to embodiments, a plurality of “parts” or “portions” may be implemented as one unit or element, or alternatively, one “part” or “portion” may include a plurality of units or elements. Hereinafter, operation principles and embodiments of the present application will be described with reference to accompanying drawings.

In the present specification, an image may include an image (hereinafter, referred to as an “oral image”) indicating at least one tooth, an oral cavity including at least one tooth, or a plaster model of an oral cavity.

Also, in the present specification, an image may include a 2-dimensional (2D) image of an object or a 3D oral image stereoscopically indicating an object. The 3D oral image may also be referred to as a 3D oral model because the 3D oral image may be generated by 3-dimensionally modeling a structure of an oral cavity, based on raw data. The 3D oral image may also be referred to as 3D scan model or 3D scan data.

Hereinafter, in the present specification, an oral image will be used as a general term for a model or image representing an oral cavity 2-dimensionally or 3-dimensionally.

Also, in the present specification, raw data may be obtained by using at least one camera to represent an object 2-dimensionally or 3-dimensionally. In detail, the raw data is data obtained to generate the oral image, and may be data (hereinafter, referred to as 2D data) obtained by at least one image sensor included in a 3D scanner when the object is canned by using the 3D scanner. The raw data may be referred to as 2D image data or 3D image data.

In the present specification, the object may be a target to be scanned. The object may be a part of a body or include a model of a part of a body. The object may include an oral cavity, an individual tooth included in an oral cavity, a plaster model or impression model of an oral cavity or an individual tooth, an artificial structure insertable into an oral cavity or an individual tooth, or a plaster model or impression model of the artificial structure. For example, the object may be at least one of teeth or gums, a plaster model or impression model of teeth or gums, and/or an artificial structure insertable into an oral cavity, or a plaster model or impression model of the artificial structure. Here, the artificial structure insertable into an oral cavity or an individual tooth may include at least one of, for example, an orthodontic appliance, an implant, a crown, an inlay, an onlay, an artificial tooth, and an orthodontic aid inserted into an oral cavity. The orthodontic appliance may include at least one of a bracket, an attachment, an orthodontic screw, a lingual orthodontic appliance, and a removable orthodontic retainer.

In the present disclosure, the object may include a region for which a die is to be designed. The region for which the die is to be designed may include a tooth region to be observed or treated. The object may include at least one region of an individual tooth, a plurality of teeth, or a gum around the individual tooth or the plurality of teeth. In addition, the object may include a plaster model of the individual tooth or the plurality of teeth. For example, the object may include a region for restoring a prosthesis.

In embodiments, the prosthesis is an artificial replacement of teeth or related tissues and may include a crown, a bridge, a partial denture, etc. The prosthesis may be manufactured based on a margin line of an abutment tooth in a maxillary base model or a mandibular base model.

The abutment tooth may refer to a tooth serving to support a prosthesis in a treatment planning stage before a fixed or removable prosthetic treatment.

In the present disclosure, the prosthesis may refer to an artificial device for protecting and restoring a defective tooth. The prosthesis may also be referred to as a crown. When one or several teeth are missing, a restoration treatment using a fixed prosthesis such as a crown or a bridge may be applied, and when the application of a fixed prosthesis is impossible or unesthetic, a removable partial denture, which is called artificial teeth, may be used to restore missing teeth. A tooth that supports a fixed or removable prosthesis is called an abutment tooth. The abutment tooth may also be referred to as a preparation tooth, or a prepared tooth.

For the purpose of orthodontic or prosthetic treatment, a die may be used among dental models. In the related art, a die for an individual tooth is generated by vertically cutting a plaster model of an oral cavity. However, a job of generating a die by using a plaster model takes a long time and a manufacturing process is cumbersome.

The disclosed embodiment is to solve the need for the above-described technology, and is to provide a technology capable of designing a die by using a three-dimensional (3D) scanner and outputting the designed die through a 3D printer.

Hereinafter, embodiments will be described in detail with reference to accompanying drawings.

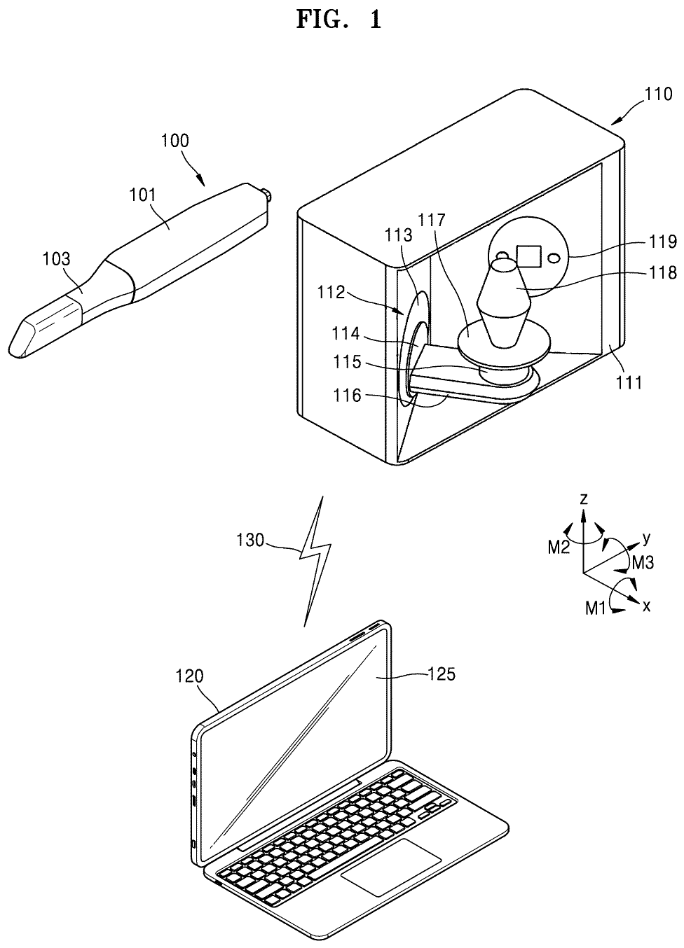

FIG. 1 is a diagram illustrating an oral image processing system according to an embodiment.

Referring to FIG. 1, the oral image processing system may include 3D scanners 100 and 110, and a data processing device 120 connected to the 3D scanner 100 and 110 through a communication network 130.

The 3D scanners 100 and 110 may be a medical device obtaining an image of an object.

The 3D scanners 100 and 110 may obtain an image of at least one of an oral cavity, an artificial structure, and a plaster model of the oral cavity or artificial structure.

The 3D scanners 100 and 110 may include at least one of an oral scanner 100 and a table scanner 110.

The oral scanner 100 may be a handheld type for scanning an oral cavity while a user holds it with a hand, and moves the oral scanner 100. The oral scanner 100 may obtain an image of an oral cavity including at least one tooth by being inserted into the oral cavity and scanning the at least one tooth in a non-contact manner.

The oral scanner 100 may include a body 101 and a tip 103. The body 101 may include a light projector (not shown) projecting light, and a camera (not shown) obtaining the image by photographing the object.

The tip 103 is a portion inserted into the oral cavity and may be detachably mounted on the body 101. The tip 103 may include a light path changer to direct the light projected from the body 101 to face the object and direct light received from the object to face the body 101.

The oral scanner 100 may obtain, as raw data, surface information of the object so as to obtain an image of at least one surface from among a tooth, a gum, and an artificial structure (e.g., an orthodontic appliance including a bracket and a wire, an implant, an artificial tooth, or an orthodontic aid inserted into an oral cavity) insertable into the oral cavity.

According to an embodiment, the 3D scanner 100 and 110 may include the table scanner 110. The table scanner 110 may be a scanner obtaining, as raw data, surface information about an object 118 by scanning the object 118 by using rotation of a table 135. The table scanner 110 may scan a surface of the object 118, such as a plaster model or impression model of an oral cavity, an artificial structure insertable into an oral cavity, or a plaster model or impression model of the artificial structure.

The table scanner 110 may include an internal space provided by being dented in an inner direction of a housing 111. The object 118 may be placed in a side surface of the internal space and a moving portion 112 capable of moving the object 118 may be formed therein. The moving portion 112 may move in an up-and-down direction along a z-axis direction. The moving portion 112 may include a fixed base 113 connected to a first rotating portion 114, the first rotating portion 114 rotatable in a first rotating direction M1 based on one point on the fixed base 113 as a center axis, for example, an x-axis as the center axis, and a beam portion 116 connected to and protruding from the first rotating portion 114. The beam portion 116 may extend or be shortened in an x-axis direction.

A second rotating portion 115 having a cylindrical shape and rotatable in a second rotating direction M2 using a z-axis as a rotating axis may be connected to the other end of the beam portion 116. The table 117 rotating together with the second rotating portion 115 may be provided on one surface of the second rotating portion 115.

An optical unit 119 may be provided on the internal space. The optical unit 119 may include a light projector projecting pattern light to the object 118, and at least one camera obtaining a plurality of 2D frames by using light reflected from the object 118. The optical unit 119 may further include a second rotating portion (not shown) rotating using the center of the light projector as a rotating axis while being combined to the side surface of the internal space. The second rotating portion may rotate the light projector and the at least one camera in a third rotating direction M3.

The 3D scanners 100 and 110 may transmit the obtained raw data to the data processing device 120 through the communication network 130.

The data processing device 120 may be connected to the 3D scanner 100 and 110 through the communication network 130 wirelessly or via wires. The data processing device 120 may be any electronic device capable of receiving the raw data from the 3D scanners 100 and 110, and generating, processing, displaying, and/or transmitting an oral image, based on the received raw data. For example, the data processing device 120 may be a computing device, such as a smartphone, a laptop computer, a desktop computer, a personal digital assistant (PDA), or a tablet personal computer (PC), but is not limited thereto. Also, the data processing device 120 may be present in the form of a server (or a server device) for processing the oral image.

The data processing device 120 may generate a 3D oral image by processing 2D image data or generate additional information, based on the 2D image data received from the 3D scanners 100 and 110. The data processing device 120 may display the 3D oral image and/or the additional information through a display 125, or output or transmit the same to an external device.

As another example, the 3D scanners 100 and 110 may obtain the raw data through an oral scan, generate 3D data by processing the obtained raw data, and transmit the 3D data to the data processing device 120.

According to an embodiment, the 3D scanners 100 and 110 may obtain the 3D data indicating a shape of the object by using the principle of triangulation according to a change in a pattern, by projecting pattern light onto the object and scanning the object onto which the pattern light is projected.

According to an embodiment, the 3D scanners 100 and 110 may obtain the 3D data of the object by using a confocal method. The confocal method is a non-destructive optical imaging technique for 3D surface measurement, and may obtain an optical cross-sectional image with a high spatial resolution by using a pinhole structure. The 3D scanners 100 and 110 may obtain the 3D data by stacking 2D images obtained along an axis direction.

However, this is only an embodiment, and the 3D scanners 100 and 110 may obtain the 3D data from raw data by using various methods in addition to the described method, and transmit the 3D data to the data processing device 120. The data processing device 120 may analyze, process, display, and/or transmit the received 3D data.

In an embodiment, the data processing device 120 may obtain a plurality of 3D oral images. A 3D oral image may also be referred to as 3D scan data or a scan model.

In an embodiment, the data processing device 120 may design a die by using 3D scan data of an object.

In an embodiment, the data processing device 120 may identify a boundary line selected for generation of the die from the 3D scan data of the object.

In an embodiment, the data processing device 120 may identify a target plane in a base model.

In an embodiment, the data processing device 120 may design the die of the object by connecting the boundary line to the target plane.

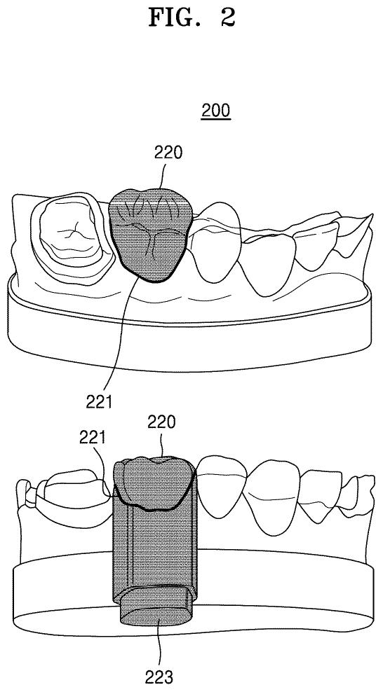

FIG. 2 is a diagram illustrating a data processing device 120 designing a die by using three-dimensional (3D) scan data, according to an embodiment.

Referring to FIG. 2, the data processing device 120 may obtain 3D scan data 200. In FIG. 2, the 3D scan data 200 may be scan data for a base model. In FIG. 2, the base model included in the 3D scan data 200 may be, for example, a mandibular base model.

In the present disclosure, a base model may refer to a model in which a tooth model is disposed and fixed. A maxillary base model may mean a model in which a plaster model of a maxilla, that is, an upper jaw of an oral cavity, is disposed. A mandibular base model may mean a model in which a plaster model of a mandible, that is, a lower jaw of an oral cavity, is disposed. An occlusion model may mean a model in which the maxillary base model and the mandibular base model are occluded.

In the present disclosure, the base model is not limited to the plaster model, and may be a model designed using 3D scan data for the oral cavity and output through a 3D printer.

In the present disclosure, a die may be a model of an individual tooth or a model of a plurality of teeth. The die may be disposed in the maxillary base model or the mandibular base model.

In the present disclosure, the die may be designed using 3D scan data and inserted into or separated from a base model printed through a 3D printer.

In an embodiment, the 3D scan data 200 for the base model may be obtained by the data processing device 120 scanning a plaster model, or a base model printed through a 3D printer. Alternatively, in an embodiment, the 3D scan data 200 for the base model may be virtually designed and generated by the data processing device 120. The mandibular base model designed using 3D scan data may be output through a 3D printer, etc. and used.

In FIG. 2, the 3D scan data 200 for the base model may include an object 220. In the present disclosure, the object 220 may refer to an object for which a die is to be generated. For example, the object 220 may be an individual tooth.

In an embodiment, the data processing device 120 may identify a boundary line 221 in the 3D scan data 200. In the present disclosure, a boundary line may refer to a line selected for generation of a die in 3D scan data.

In an embodiment, when a user selects the object 220, the data processing device 120 may automatically generate the boundary line 221 for the selected object 220. For example, the data processing device 120 may generate the boundary line 221 between a tooth identified as the object 220 and a gum around the tooth. In an embodiment, the data processing device 120 may generate a boundary line 221 by identifying points included in a boundary region between a tooth and a gum with respect to the object 220 selected by the user, and connecting the identified points. The user may modify or edit the boundary line 221 generated by the data processing device 120.

Alternatively, in an embodiment, the data processing device 120 may receive a selection of a position of the boundary line 221 from the user. The user may select a region or a position in which a die is to be generated. The data processing device 120 may generate the boundary line 221 by connecting points in the region or position selected by the user.

In an embodiment, the data processing device 120 may identify a target plane 223.

In an embodiment, the target plane 223 is an opposite surface of a die and may refer to a plane to be used as a bottom surface of the die. The target plane 223 may be a plane included in a bottom surface of the base model and parallel to the bottom surface of the base model.

In an embodiment, the data processing device 120 may identify the target plane 223 on the bottom surface of the base model. A position of the target plane 223 may vary depending on a direction of a projection line projected from the boundary line 221 for generation of a die. That is, an angle between a projection line and a target plane may also change according to a projection direction of the projection line connecting the boundary line and a bottom surface of the base model.

For example, a direction of the projection line may be determined such that the projection line projected from the boundary line 221 and the bottom surface of the base model are orthogonal, and the direction of the projection line may be determined such that the projection line projected from the boundary line 221 and the bottom surface of the base model meet at an angle of 120 degrees.

In an embodiment, the data processing device 120 may receive, from a user, a selection of a direction of the projection line projected from the boundary line 221 to the base model for generation of a die. Alternatively, the data processing device 120 may determine a direction of the projection line projected from the boundary line 221 to the base model in a preset direction. Alternatively, the data processing device 120 may automatically identify a projection direction in which the projection line meets an interference region to a minimum.

In an embodiment, the data processing device 120 may identify a region in which the projection line meets the bottom surface of the base model as a target plane by projecting the projection line from the boundary line 221 in the determined direction of the projection line.

In an embodiment, the data processing device 120 may design the die of the object 220 by connecting the boundary line 221 to the target plane 223.

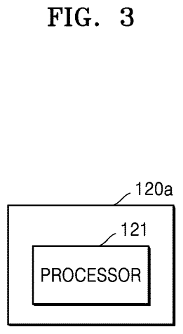

FIG. 3 is an internal block diagram of a data processing device according to an embodiment.

A data processing device 120a of FIG. 3 may be an embodiment of the data processing device 120 of FIG. 1. Details redundant those described with reference to the data processing device 120 of FIG. 1 are omitted.

In an embodiment, the data processing device 120a may also be referred to as an oral image processing apparatus.

Referring to FIG. 3, the data processing device 120a may include a processor 121.

In an embodiment, one or a plurality of processors 121 may be present. The processor 121 may perform at least one instruction to control at least one component included in the data processing device 120a such that an intended operation is performed.

Here, the at least one instruction may be stored in a memory (not shown) included in the data processing device 120a or an internal memory (not shown) included in the processor 121 separately from the processor 121.

In an embodiment, the at least one instruction may include an instruction for executing dedicated software for designing a die of an object based on scan data.

In an embodiment, the processor 121 may execute one or more instructions to obtain 3D scan data of an object.

In an embodiment, the processor 121 may design a die of an object by using 3D scan data of the object.

In an embodiment, the processor 121 may identify a boundary line selected for generation of a die from 3D scan data of an object. The boundary line may refer to a line selected from 3D scan data for generation of a die.

In an embodiment, the processor 121 may generate a boundary line at a selected position by receiving a selection of a boundary line position from a user, or may automatically generate a boundary line in a region around a selected object.

In an embodiment, the processor 121 may identify a direction of a projection line to be projected from a boundary line to a base model.

In an embodiment, the processor 121 may identify a region in which the projection line meets the bottom surface of the base model as a target plane by projecting the projection line from the boundary line to the base model in the direction of the identified projection line.

In an embodiment, the processor 121 may design a die of an object by connecting the boundary line to the target plane included in the bottom surface of the base model.

In an embodiment, the processor 121 may design a die by vertically connecting the boundary line to the target plane. When the projection direction of the projection line and the bottom surface of the base model are orthogonal, the processor 121 may obtain a projection line that is projected at a point of the boundary line and vertically meets the target plane, by using the target plane as a plane of projection. In an embodiment, the processor 121 may design a die of an object by using a curved surface surrounded by the boundary line and a figure surrounded by the projection line projected vertically to the target plane.

In an embodiment, the processor 121 designing a die of an object may mean a 3D die model formatively before manufacturing the die into a product. The die model designed by the processor 121 may be manufactured as a product by a 3D printer or a miller and used. The die model manufactured into the product may be disposed in a detachable form on a base model manufactured through a 3D printer, etc. and used.

In an embodiment, the processor 121 may execute one or more instructions to identify whether the projection line projected from the boundary line to the target plane meets an interference region that is part of scan data of an object. In an embodiment, the interference region may be a region in which the projection line projected from the boundary line meets before meeting a plane of projection. The interference region may be a partial region of the curved surface surrounded by the boundary line.

In an embodiment, the processor 121 may obtain a projection diagram formed by the projection line vertically meeting the plane of projection in order to identify whether the projection line meets the interference region. In an embodiment, when two or more single closed curves are included in the projection diagram, the processor 121 may identify that the projection line meets the interference region. In an embodiment, the processor 121 may design a die of an object by using a projection line projected only at points on the boundary line corresponding to the longest single closed curve among the two or more single closed curves, based on the projection diagram including two or more single closed curves. A point of the boundary line corresponding to a single closed curve may mean a point in the boundary line connected to the single closed curve by the projection line.

When a die is designed for an object by using a projection line projected only at points on the boundary line corresponding to the longest single closed curve among two or more single closed curves, there is an empty space between a boundary line corresponding to the remaining single closed curve rather than the longest single closed curve and the interference region.

In an embodiment, the processor 121 may execute one or more instructions to fill the space between a point of the boundary line corresponding to the remaining single closed curve and the interference region with a mesh. In an embodiment, the processor 121 may generate a mesh by connecting the point of the boundary line corresponding to the remaining single closed curve excluding the longest single closed curve to a point on the projection line at the shortest distance from the point of the boundary line corresponding to the remaining single closed curve. In an embodiment, the processor 121 may prevent an empty space from occurring in the die by filling, with the mesh, the empty space between the interference region and the point of the boundary line corresponding to the remaining single closed curve.

In an embodiment, the processor 121 may execute one or more instructions to display a designed die.

In an embodiment, when the projection diagram includes two or more single closed curves, the processor 121 designs the die of the object by using the projection line projected only at the points on the boundary line corresponding to the longest single closed curve, and thus, a cross section of the designed die includes only one single closed curve.

In an embodiment, the processor 121 may design a die so that meshes included in an upper surface and a side surface of the die have the same or similar lengths. That is, the processor 121 may allow meshes of the same length to be included in the upper surface of the die, that is, the curved surface surrounded by the boundary line and in the side surface of the die, that is, a curved surface formed by a mold surrounded by the projection line connecting the boundary line to the target plane. To this end, in an embodiment, the processor 121 may obtain a length of an average side of meshes included in the curved surface surrounded by the boundary line. In an embodiment, the processor 121 may obtain a difference distance between a point of the boundary line located at the farthest distance from the target plane and a point of the boundary line located at the closest distance from the target plane, divide the difference distance by the length of the average side of meshes, and obtain the number N (N is a natural number) of partial layers.

In an embodiment, the processor 121 may design a die by forming N partial layers having the length of the average side of meshes between the farthest point of the boundary line and the closest point of the boundary line. In this case, because the length of the partial layer is the length of the side of the mesh, the mesh included in the upper surface of the die and the mesh included in the side of the die have the same size.

In an embodiment, the processor 121 may simultaneously or sequentially form the N partial layers.

In an embodiment, in order to sequentially form partial layers, the processor 121 may form a first partial layer below the farthest point of the boundary line from the target plane, with a projection line having the length of the average side of meshes while being projected from the farthest point of the boundary line from the target plane toward the target plane.

In an embodiment, the processor 121 may obtain a new boundary line by connecting a line of the first partial layer to a boundary line below which the first partial layer is not formed. In an embodiment, the processor 121 may sequentially form a second partial layer below the first partial layer, with a projection line projected from a point of the new boundary line toward the target plane and having the length of the average side of meshes.

In an embodiment, when obtaining a new boundary line, the processor 121 may determine whether there is an interference region between the new boundary line and the plane of projection. In an embodiment, when two or more single closed curves are included in the projection diagram formed by the projection line projected from the point of the new boundary line, the processor 121 may form a second partial layer by using a projection line projected only at points of the new boundary line corresponding to the longest single closed curve among the two or more single closed curves.

As described above, according to an embodiment, the processor 121 may generate a die by using the projection line projected from the boundary line toward the target plane.

In another embodiment, the processor 121 may design a die by using the first projection line and the second projection line by projecting the first projection line in a direction of the first plane of projection from the boundary line, and projecting again the second projection line in a direction of the target plane from the first plane of projection.

To this end, in an embodiment, the processor 121 may obtain a first plane of projection.

In an embodiment, the processor 121 may obtain a convex hull curved surface of a boundary line.

In two-dimensional (2D) space, a convex hull may refer to a convex polygon having a minimum size including all of a plurality of points on a plane. In the 2D space, the convex hull is a polygon formed by a closed curve, whereas in 3D space, the convex hull has a shape of a closed curved.

In an embodiment, the processor 121 may obtain a normal vector direction of the convex hull curved surface. In an embodiment, the processor 121 may obtain, as the first plane of projection, a plane that has a slope perpendicular to a normal vector direction and is in contact with the boundary line or is the shortest distance away from the boundary line among planes spaced apart from the boundary line.

In an embodiment, the processor 121 may obtain a weighted normal vector direction to which a weight is added according to a mesh area in consideration of a weight according to the area of a mesh forming the convex hull curved surface.

In an embodiment, the processor 121 may obtain a first projection line that is projected from a point of the boundary line and vertically meets the first plane of projection. In an embodiment, the processor 121 may obtain the second projection line that is projected from a point where the first projection line and the first plane of projection meet and vertically meets the second plane of projection by using the target plane as the second plane of projection.

In an embodiment, the processor 121 may design a die by using a mold surrounded by the first projection line and the second projection line. At this time, the generated die may include a curved surface surrounded by the boundary line as an upper surface of the die, and a curved surface surrounded by the first projection line and a curved surface surrounded by the second projection line as a side surface of the die.

In an embodiment, when generating a curved surface surrounded by the first projection line, the processor 121 may identify whether an interference region is included between the first projection line and the first plane of projection.

In an embodiment, the processor 121 may identify that an interference region is included based on the fact that two or more single closed curves are included in the first projection diagram formed by the first projection line on the first plane of projection. In an embodiment, the processor 121 may generate a die by obtaining a projection line projected only from points of the boundary line corresponding to the longest single closed curve among the two or more single closed curves as the first projection line.

In an embodiment, the processor 121 may generate a mesh connecting the point of the boundary line corresponding to the remaining single closed curve to the point on the projection line at the shortest distance from the point of the boundary line corresponding to the remaining single closed curve in order to fill empty space between the interference region and the point of the boundary line corresponding to the remaining single closed curve. The processor 121 may fill, with the mesh, between the interference region and the boundary line corresponding to the remaining single closed curve.

In an embodiment, when two or more single closed curves are included in the first projection diagram formed by the first projection line on the first plane of projection, because the processor 121 designs a die of the object by using only a projection line, as the first projection line, projected from only points of the boundary line corresponding to the longest single closed curve among the two or more single closed curves, a cross-section of the designed die includes only one single closed curve.

In an embodiment, when generating a curved surface surrounded by the first projection line, the processor 121 may obtain the number N (N is a natural number) of partial layers by dividing a distance between a farthest point of the boundary line from the first plane of projection and the first plane of projection by the length of the average side of meshes included in the curved surface surrounded by the boundary line. In an embodiment, the processor 121 may form a curved surface surrounded by the first projection line by forming N partial layers having the length of the average side of meshes between the farthest point of the boundary line and the first plane of projection.

In an embodiment, the processor 121 may form a first partial layer below the farthest point of the boundary line by the projection line projected from the farthest point of the boundary line from the first projection line toward the first plane of projection and having the length of the average side of meshes. In an embodiment, the processor 121 may obtain a new boundary line by connecting a line of the first partial layer to a boundary line below which the first partial layer is not formed, and form a second partial layer below the first partial layer, with a projection line projected from a point of the new boundary line toward the first plane of projection and having the length of the average side of meshes. As such, the processor 121 may form a curved surface surrounded by the first projection line including the N partial layers.

In an embodiment, the processor 121 may form the second partial layer by using the projection line projected only from points of the new boundary line corresponding to the longest single closed curve among two or more single closed curves based on the fact that the two or more single closed curves are included in a projection diagram formed by the projection line projected from the point of the new boundary line on the first plane of projection.

In an embodiment, the processor 121 may obtain the number M (M is a natural number) of partial layers by dividing the maximum distance among vertical distances between the first plane of projection and the second plane of projection by the length of the average side of meshes by using the target plane as the second plane of projection, and form M or fewer partial layers having the length of the average side of meshes between the first plane of projection and the second plane of projection so that the curved surface surrounded by the second projection line is a side surface of the die.

As described above, according to an embodiment, the data processing device 120a may design the die of the object by using 3D scan data of the object.

In addition, according to an embodiment, when an interference region is included between the boundary line and the target plane, the data processing device 120a may exclude the interference region and design the die with the remaining region.

In addition, according to an embodiment, the data processing device 120a may fill an empty region that is excluded as the interference region with a mesh connecting the boundary line to a point of a peripheral projection line.

In addition, according to an embodiment, the data processing device 120a may design a die by using a projection line vertically connecting the boundary line to the target plane.

In addition, according to an embodiment, the data processing device 120a may design a die by inserting another plane of projection between the boundary line and the target plane and connecting the boundary line to the plane of projection and the plane of projection to the target plane.

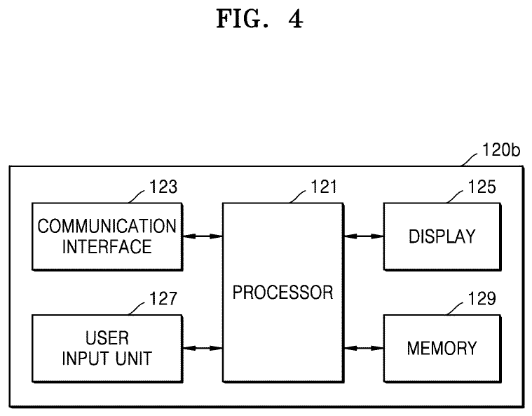

FIG. 4 is an internal block diagram of a data processing device according to an embodiment.

A data processing device 120b of FIG. 4 may be an embodiment of the data processing device 120a of FIG. 3. Details redundant those described with reference to the data processing device 120a of FIG. 3 are omitted.

Referring to FIG. 4, the data processing device 120b may further include a communication interface 123, a display 125, a user input unit 127, and a memory 129 in addition to the processor 121.

In an embodiment, the processor 121 may execute one or more instructions to design a die of an object by using 3D scan data of the object.

The data processing device 120b may generate, process, display, and/or transmit a 3D oral model, by using raw data and/or 3D information received from the 3D scanners 100 and 110. Alternatively, the data processing device 120b may receive a 3D oral model through a wired or wireless communication network from an external server or an external device.

In an embodiment, the processor 121 may perform at least one instruction to control one or more components included in the data processing device 120b so that an intended operation is performed. Therefore, even though a case where the processor 121 performs certain operations is described as an example, it may mean that the processor 121 controls one or more components included in the data processing device 120b so that certain operations are performed.

The communication interface 123 according to an embodiment may communicate with at least one external electronic device through a wired or wireless communication network.

For example, the communication interface 123 may communicate with the 3D scanner 110 by the control of the processor 121. In an embodiment, the communication interface 123 may receive raw data or obtain 3D information from the 3D scanner 110. In an embodiment, the communication interface 123 may obtain a scan model by communicating with an external electronic device or an external server other than the 3D scanner 110.

The communication interface 123 may include at least one short-range communication module performing communication according to the communication standard, such as Bluetooth, Wi-Fi, Bluetooth low energy (BLE), near field communication (NFC)/radio frequency identification (RFID), Wi-Fi direct (WFD), ultra-wideband (UWB), or ZigBee.

In addition, the communication interface 123 may further include a long-range communication module performing communication with a server for supporting long-range communication according to the long-range communication standard. Specifically, the communication interface 123 may include the long-range communication module performing communication through a network for Internet communication. For example, the communication interface 123 may include the long-range communication module performing communication through a communication network according to the communication standard, such as third generation (3G), fourth generation (4G), and/or fifth generation (5G).

In addition, the communication interface 123 may communicate with the 3D scanner 110, an external server, or an external electronic device by wired. To this end, the communication interface 123 may include at least one port to be connected to the 3D scanner 110 or the external electronic device through a wired cable. The communication interface 123 may communicate with the 3D scanner 110 or the external electronic device connected via wires through the at least one port.

In an embodiment, the communication interface 123 may transmit a designed die to an external electronic device or an external server. For example, the communication interface 123 may transmit the designed die to a 3D printer or a miller.

The display 125 according to an embodiment may output 3D scan data. In an embodiment, the display 125 may output 3D scan data to be used to design a die. In an embodiment, the display 125 may output a boundary line being displayed for an object included in the 3D scan data. In an embodiment, the display 125 may output a die being designed by connecting a boundary line to a target plane.

The user input unit 127 according to an embodiment may receive a user input for controlling the data processing device 120b. The user input unit 127 may include a touch panel for detecting a touch of a user, a button for receiving push manipulation of the user, and a user input device including a keyboard or a mouse for designating or selecting one point on a user interface screen, but is not limited thereto. In addition, the user input unit 127 may include a voice recognition device for voice recognition. For example, the voice recognition device may be a microphone and may receive a user's voice command or voice request. Accordingly, the processor 121 may control an operation corresponding to the voice command or the voice request to be performed.

In an embodiment, the user input unit 127 may receive a command to design a die.

In an embodiment, the user input unit 127 may receive a selection of 3D scan data of an object for which a die is to be designed from a user.

In an embodiment, the user input unit 127 may receive a selection of an object to design a die from a user such as a dentist, etc.

In an embodiment, the user input unit 127 may receive information for generation of a die from the user. The information for generation of the die may include, for example, information about a boundary line for designing the die, a projection direction of a projection line connecting the die to the target plane, whether to design the die by using only one projection direction, or whether to design the die by using a plurality of projection directions.

In an embodiment, the user input unit 127 may receive, from the user, a selection of a position of the boundary line or a command to automatically generate the boundary line. Alternatively, the user input unit 127 may receive a command for modifying or editing a generated boundary line?.

In an embodiment, the user input unit 127 may receive a selection of a projection direction of a projection line projected from the boundary line from the user. Alternatively, the user input unit 127 may receive a selection of a position of the target plane from the user.

In an embodiment, the user input unit 127 may receive from the user a selection of whether to design the die by using only a projection line projected from the boundary line to the target plane, or by using a projection line projected from the boundary line to another plane of projection by using another plane of projection between the boundary line and the target plane and a projection line projected from another plane of projection to the target plane together.

In an embodiment, the user input unit 127 may receive from the user a selection of whether a plurality of partial layers are formed at once or sequentially formed between the boundary line and the plane of projection.

The memory 129 according to an embodiment may store at least one instruction. The memory 129 may store at least one instruction or program executed by the processor 121.

In an embodiment, the memory 129 may store data received from the 3D scanner 110, for example, raw data or 3D information obtained by scanning an oral cavity or an oral model. The memory 129 may store position information of points of 3D oral data and connection relationship information between the points which are received from the 3D scanner 110. In addition, the memory 129 may store a 3D oral model generated by the data processing device 120b, received from the 3D scanner 110, or received from an external server or an external device.

In an embodiment, the memory 129 may store and execute dedicated software interoperating with the 3D scanner 110. Here, the dedicated software may be referred to as a dedicated program or a dedicated application. When the data processing device 120b operates in conjunction with the 3D scanner 110, dedicated software stored in the memory 129 may be connected to the 3D scanner 110 to in real time receive data obtained through scan of an object. The dedicated software may provide a user interface for using data obtained from the 3D scanner 110 through the display 125. A screen of the user interface provided through the dedicated software may include 3D scan data of an object.

In an embodiment, the memory 129 may store a plurality of 3D oral models.

In an embodiment, the 3D oral model may include 3D scan data of an object.

In an embodiment, identification information for 3D scan data may be stored in the memory 129. The identification information for the 3D scan data may be information indicating a type of the 3D scan data. The identification information for the 3D scan data may include information indicating whether the 3D scan data is about the maxilla or the mandible, and information indicating which tooth relates to the 3D scan data. The identification information for the 3D scan data may be stored in the memory 129 together with the 3D scan data in the form of an index, a tag, or a file name for the 3D scan data.

In an embodiment, the memory 129 may include one or more instructions for designing a die of the object based on the 3D scan data of the object.

In an embodiment, dedicated software for designing the die of the object based on the 3D scan data of the object may be stored in the memory 129. The dedicated software for designing the die may be referred to as a dedicated program, a dedicated tool, or a dedicated application. The dedicated software may design the die based on the 3D scan data of the object.

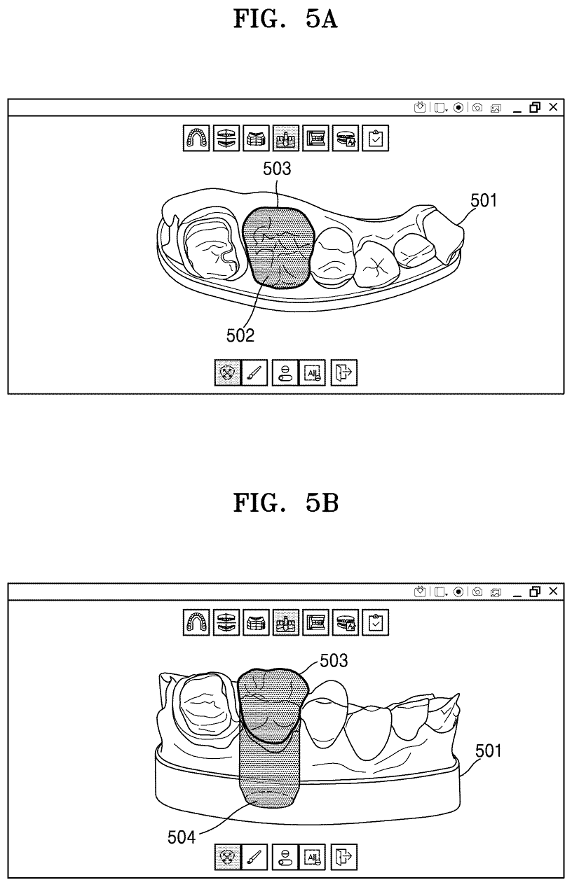

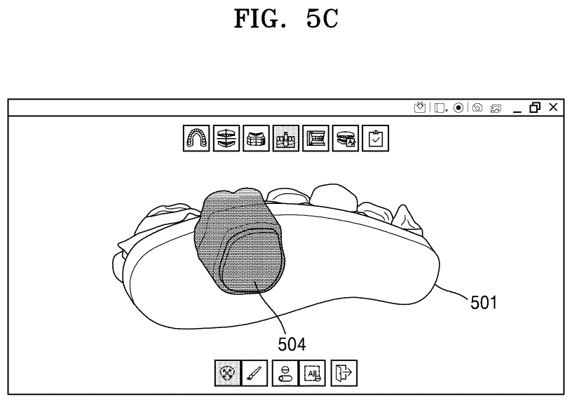

FIGS. 5A, 5B, and 5C are diagrams illustrating the data processing device 120 designing a die of an object, according to an embodiment.

A user may select the object for which the die is to be designed or select 3D scan data of the object. The data processing device 120 may output, on a screen, 3D scan data generated for the object selected by the user.

In an embodiment, the 3D scan data of the object may be scan data including only data of an individual tooth to generate a die and a gum around the individual tooth. Alternatively, the 3D scan data of the object may be scan data including data of adjacent teeth and adjacent gums in addition to the individual tooth for which the die is to be generated, and the gum around the individual tooth.

In an embodiment, the 3D scan data of the object may be scan data obtained by scanning an oral cavity including the object, or scan data obtained by scanning an oral cavity model including an object model.

Referring to FIG. 5, the data processing device 120 may output a screen designing the die of the object.

In an embodiment, FIG. 5A illustrates the data processing device 120 outputting a 3D scan data 501 on a screen. FIG. 5A illustrates the 3D scan data 501 of a top-down view of a base model including an object 502.

Referring to FIG. 5A, it may be seen that the 3D scan data 501 includes data of adjacent teeth around the object 502 in addition to the object 502. However, this is an embodiment, for example, when the object 502 is an individual tooth, the 3D scan data 501 may include only scan data for the individual tooth or may include only scan data for a gum adjacent to the individual tooth.

FIG. 5A illustrates the 3D scan data 501 obtained by scanning an oral model, for example, a mandibular base model. However, the 3D scan data 501 is not limited thereto, and may be data obtained by the data processing device 120 designing a mandibular base model based on raw data.

In an embodiment, the user may select an object for which a die is to be generated. In an embodiment, the user may select the object 502 by using a boundary line 503.

In an embodiment, the boundary line 503 may refer to a boundary line of the object 502 selected for generation of a die. The boundary line 503 may be a line used to separate a tooth from a gum, but is not limited thereto. For example, when the user wants to generate a die for only a partial region of the tooth, the user may select a line that separates a part of the tooth from the remaining tooth as a boundary line. In addition, when the user wants to generate a die for a region including the tooth and an adjacent gum, the user may select a line that separates the tooth and the adjacent gum from the remaining region as a boundary line. The user may select a position of the boundary line 503 by selecting points around a region where the die is to be generated. The data processing device 120 may generate the boundary line 503 by connecting points selected by the user.

The user may select an object by using a method of pointing the object 502 for which the die is to be generated from among the 3D scan data 501 output on the screen. The data processing device 120 may identify the object 502 selected by the user and automatically generate the boundary line 502 around the selected object 502.

Alternatively, the user may select the object 502 by inputting a unique identification number of the object 502, for example, a tooth number. The data processing device 120 may identify the object 502 having the tooth number selected by the user from the 3D scan data 501. The data processing device 120 may automatically generate a boundary line around the object 502 selected by the user.

In an embodiment, the data processing device 120 may overlap and output the boundary line 503 on the 3D scan data 501 as shown in FIG. 5A.

In an embodiment, the data processing device 120 may receive a die generation request from the user and identify a target plane 504 corresponding thereto. In an embodiment, the target plane 504 may mean a plane on which the opposite surface of the die is to be formed, that is, a plane on which a bottom surface of the die is to be formed.

In an embodiment, the data processing device 120 may identify the target plane 504 parallel to a bottom surface of the base model and included in the bottom surface of the base model.

In an embodiment, the data processing device 120 may project a projection line from the boundary line 503 as a base model by receiving a selection of a projection direction for generation of a die from the user, using a preset projection direction, or using a projection direction in which an interference region between the boundary line 503 and a plane of projection is minimized. In an embodiment, the data processing device 120 may identify, as the target plane 504, a region projected from the boundary line 503 and meeting the bottom surface of the base model.

In an embodiment, the data processing device 120 may project the projection line such that the projection line from the boundary line 503 vertically crosses the bottom of the base model, and identify, as the target plane 504, a region in which the projection line meets the bottom of the base model.

Projection may mean projecting a shape of an object onto a plane. A plane in which the shape of an object appears through projection is called a plane of projection, and a line extending the object and the plane of projection is called a projection line.

In an embodiment, the data processing device 120 may project a projection line such that a projection line projected from each of a plurality of points forming the boundary line 503 meets the bottom surface of the base model. For example, the data processing device 120 may project a projection line such that a projection line projected from each of a plurality of points forming the boundary line 503 vertically meets the bottom surface of the base model.

In an embodiment, the data processing device 120 may form the target plane 504 by connecting points where the projection line projected from the boundary line 503 meets the base model, and design a die by using the projection line connecting the boundary line 503 to the target plane 504.

In an embodiment, the data processing device 120 may design the die of the object 502 by using a curved surface surrounded by the boundary line 503, that is, a curved surface formed on the boundary line 503 as an upper surface of the die, and a mold surrounded by the plane of projection as a side surface of the die.

FIG. 5B illustrates the 3D scan data 501 when a base model including the object 503 is viewed from the side. The data processing device 1200 may overlap and output a designed die on the 3D scan data 501 as shown in FIG. 5B,

FIG. 5C illustrates the 3D scan data 501 when a base model including the object 503 is viewed from below. As shown in FIG. 5C, it may be seen that the target plane 504, which is the bottom surface of the die, is included in the bottom of the base model.

As described above, according to an embodiment, the data processing device 120 may design the die 505 by using the 3D scan data 501 for the object 502. The die designed by the data processing device 120 may be generated as a product and used for a base model, etc.

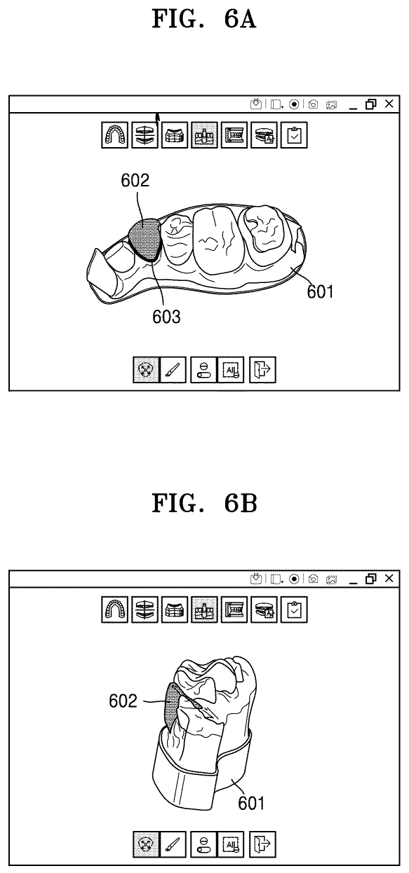

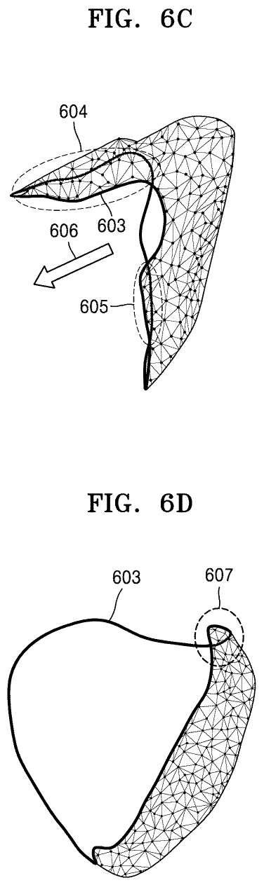

FIGS. 6A, 6B, 6C, and 6D are diagrams illustrating a case where an interference region is included when the data processing device 120 designs a die of an object, according to an embodiment.

The data processing device 120 may output a screen designing a die of an object.

FIG. 6A shows 3D scan data 601 outputting of a top-down view of a base model. Referring to FIG. 6A, the data processing device 120 may overlap and display a boundary line 603 on the 3D scan data 601.

FIG. 6B illustrates the 3D scan data 601 when the same base model as the base model shown in FIG. 6A is viewed from the side.

FIGS. 6C and 6D illustrate only an upper region of an object 602, that is, a curved surface of the object 602 surrounded by a boundary line 603, separated from a base model. When the curved surface surrounded by the boundary line 603 is referred to as, for example, a target curved surface, target curved surfaces shown in FIGS. 6C and 6D corresponds to a case where the same target curved surfaces as the target curved surfaces shown in FIGS. 6A and 6B are enlarged and viewed from a different angle.

Referring to FIGS. 6A to 6D, it may be seen that the target curved surfaces have various shapes depending on viewing angles. Therefore, a projection diagram formed by a projection line projected from the boundary line 603 of the target curved surface meeting a plane of projection also varies depending on a projection direction.

In this disclosure, a curved surface formed by a boundary line selected for generation of a die is an opened curved surface other than a closed curved surface. When a projection line is projected in a specific direction from a boundary line, which is a boundary line of an opened curved surface, the projection line may first meet an interference region before meeting a plane of projection according to a projection direction. Here, the interference region is a region where the projection line projected from the boundary line meets before meeting the plane of projection, and may mean a region included between the boundary line and the plane of projection. The interference region may be a partial region of an opened curved surface surrounded by a boundary line.

For example, when a projection line is projected in a specific projection direction, e.g., in a direction indicated by reference numeral 606, on the boundary line 603 of the target curved surface shown in FIG. 6C, the projection line projected from the point of the boundary line 603 does not meet the interference region before being projected onto the plane of projection. However, assuming that the projection line is projected forward, i.e., in a front direction, on the boundary line 603 of a target curved surface shown in FIG. 6C, a line of the boundary lines 603 included in regions denoted by reference numerals 604 and 605 meet the target curved surface before being projected from a point of the line to a plane of projection located in the front direction.

Similarly, when a projection line is projected in the front direction from the boundary line 603 of a target curved surface shown in FIG. 6D, a rear line of the boundary lines 603 included in a region denoted by reference numeral 607 meets a front curved surface before being projected from a point included in the rear line to a plane of projection located in the front direction. That is, an interference region is included between the rear boundary line located in the region denoted by reference numeral 607 and the plane of projection.

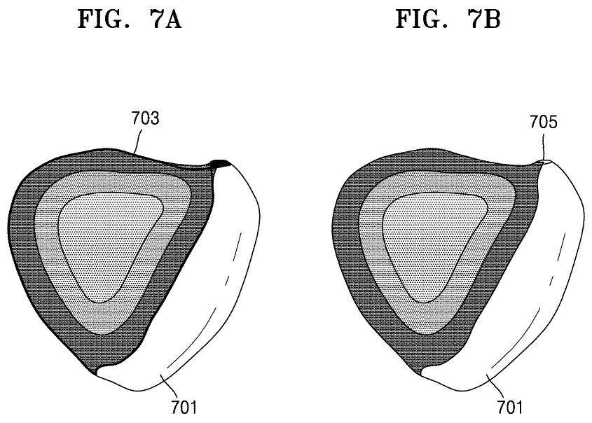

FIGS. 7A and 7B are diagrams illustrating the data processing device 120 generating a die by removing an interference region, according to an embodiment.

FIGS. 7A and 7B illustrate resultants obtained when the data processing device 120 generates a die by projecting a projection line in a front direction on the boundary line 603 of a target curved surface shown in FIG. 6D.

When a projection direction is front, because a target plane, that is, a plane of projection is located in front, the data processing device 120 connects a boundary line 703 and the target plane by using a projection line projected from the boundary line 703 in a front direction, and designs a die by using the connected boundary line 703 and target plane.

As described in FIG. 6D, the boundary lines 603 and 703 of the target curved surface shown in FIGS. 6D and 7 each include a region that meets an interference region when the projection line is projected in the front direction.

Even though the interference region is included between the boundary line 703 of the target curved surface and the plane of projection, when the data processing device 120 designs a die without considering the interference region, a projection line meeting the interference region among projection lines is also projected in a projection direction, which may generate a die in a shape that a user does not want.

Accordingly, in an embodiment, the data processing device 120 may identify a projection line meeting the interference region. In an embodiment, the data processing device 120 may design a die by using only a projection line that does not meet the interference region, excluding the projection line meeting the interference region.

FIGS. 7A and 7B illustrate a die 701 generated when the data processing device 120 removes the projection line meeting the interference region and designs a die by using only the remaining projection line, according to an embodiment.

FIG. 7A shows a case where the boundary line 703 is marked on the die 701, and FIG. 7B shows a case where the boundary line 703 is not marked on the die 701.

As shown in FIG. 7, when the data processing device 120 excludes the projection line that meets the interference region and designs the die by using only the projection line that does not meet the interference region, an empty space denoted by reference numeral 705 is formed between the boundary line 703 and the interference region. That is, when the data processing device 120 excludes the projection line meeting the interference region, because data corresponding to the excluded projection line is excluded, the empty space 705 having no data is generated in the die 701 generated while the projection line meeting the interference region is excluded.

In an embodiment, the data processing device 120 may fill the empty space 705 by using information about points included in a peripheral line. For example, the data processing device 120 may generate a triangular mesh by connecting points included in a line surrounding the empty space 705 to each other. The data processing device 120 may fill the empty space 705 by using the generated mesh.

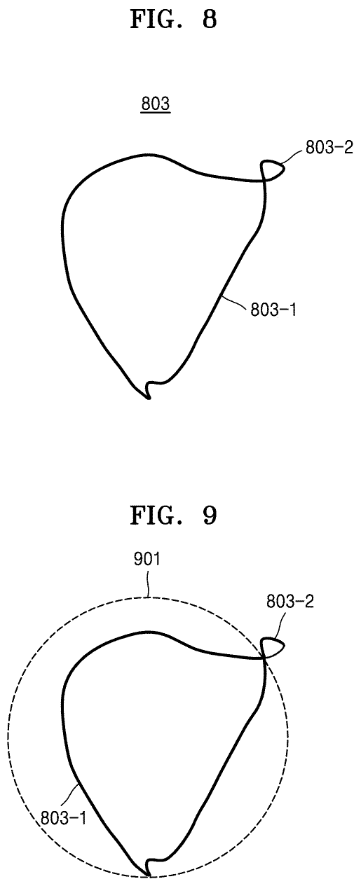

FIG. 8 is a diagram illustrating the data processing device 120 identifying whether there is an interference region when generating a die, according to an embodiment.

In an embodiment, the data processing device 120 may determine whether there is the interference region between a boundary line and a plane of projection before generating the die.

In an embodiment, the data processing device 120 may determine whether there is the interference region by using a projection diagram formed by a projection line on the plane of projection.

The projection diagram drawn by the projection line on the plane of projection has various forms depending on a projection angle.

A projection line projected from a boundary line of an opened curved surface may first meet an interference region before meeting a plane of projection according to a projection direction. In the present disclosure, the interference region may mean a region that meets a projection line projected from a boundary line to a plane of projection.

When no interference region is included between the boundary line and the plane of projection, a projection diagram generated when the projection line from the boundary line meets the plane of projection includes only one single closed curve. A single closed curve is a closed curve in which a starting point and an ending point of a curve coincide, and refers to a curve that continuously one-on-one corresponds to one circumference on a plane. The single closed curve may have a circumferential shape of a circle or a shape in which a circumference is deformed.

On the other hand, when the interference region is included between the boundary line and the plane of projection, the projection diagram generated when the projection line from the boundary line meets the plane of projection includes a plurality of single closed curves.

FIG. 8 is a projection diagram of a projection line projected in the front direction from the boundary line 603 of the target curved surface shown in FIG. 6D drawn on a plane of projection located in the front direction. Referring to FIG. 8, it may be seen that the projection diagram is drawn in the form of a curve 803 in which two single closed curves 803-1 and 803-2 are connected. That is, when an interference region is included between the boundary line and the plane of projection, the projection diagram drawn by the projection line from the boundary line may be in the shape of a polyline in which two or more single closed curves are connected into one.

The projection diagram shown in FIG. 8 is in the shape in which two single closed curves 803-1 and 803-2 are connected into one, but this is an embodiment in which when the interference region is included between the boundary line and the plane of projection, the projection diagram drawn by the projection line from the boundary line on the plane of projection may have a curved shape in which three or more single closed curves are connected to each other.

In an embodiment, when the two or more single closed curves 803-1 and 803-2 are included in the projection diagram as shown in FIG. 8, the data processing device 120 may determine that there is the interference region between the boundary line and the plane of projection.

FIG. 9 is a diagram illustrating the data processing device 120 removing an interference region, according to an embodiment.

A projection diagram shown in FIG. 9 is the same projection diagram as the projection diagram shown in FIG. 8.

In an embodiment, when two or more single closed curves are included in the projection diagram and it is determined that there is an interference region between a boundary line and a plane of projection, the data processing device 120 may remove the remaining single closed curve while leaving only one of two or more single closed curves.

In an embodiment, the data processing device 120 may identify a single closed curve 803-1 having the longest length among single closed curves 803-1 and 803-2 included in the projection diagram shown in FIG. 9.

In an embodiment, the data processing device 120 may select only the single closed curve 803-1 having the longest length and remove the remaining single closed curve 803-2.

In an embodiment, when a plurality of single closed curves included in the projection have the same length, the data processing device 120 may select only one of the plurality of single closed curves by using a normal vector of scan data and remove the remaining single closed curves. For example, the data processing device 120 may obtain a normal vector direction of a plurality of single closed curves and select a single closed curve having the same direction as the normal vector direction of the target curved surface. However, the data processing device 120 is not limited thereto, and may select only one of a plurality of single closed curves by using various methods.

In an embodiment, the data processing device 120 may obtain a loop 901 entirely surrounding the selected single closed curve 803-1.

In an embodiment, the data processing device 120 may identify a boundary line corresponding to the single closed curve 803-1 included in the loop 901. The boundary line corresponding to the single closed curve 803-1 may mean a boundary line connected to the single closed curve 803-1 by a projection line.

In an embodiment, the data processing device 120 may design a die of an object by using projection lines projected from points on the boundary line corresponding to the single closed curve 803-1 included in the loop 901.

Accordingly, according to an embodiment, the data processing device 120 may design a die in the shape intended by the user by identifying whether there is an interference region when generating a die, and removing the interference region when there is the interference region.

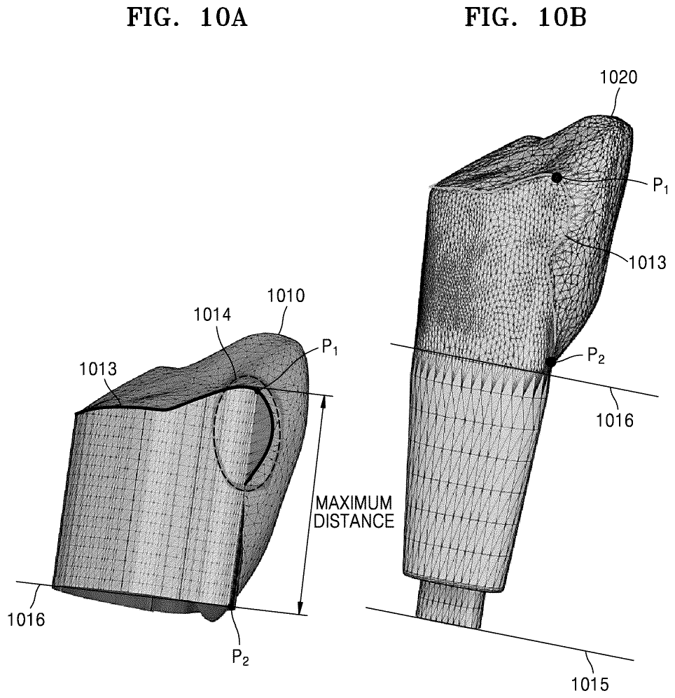

FIG. 10 is a diagram illustrating the data processing device 120 designing a die of an object, according to an embodiment.

A target curved surface 1010 shown in FIG. 10 may be the same as the target curved surface shown in FIG. 6. Therefore, the descriptions of FIGS. 6 to 9 may also be applied to the target curved surface 1010 shown in FIG. 10.

Referring to FIG. 10, the data processing device 120 may obtain 3D scan data of an object and identify a boundary line 1013 selected for generation of a die from 3D scan data of the object.

In an embodiment, the data processing device 120 may design a die by projecting a projection line from a point of the boundary line 1013 to a target plane 1015, by using the target plane 1015 included in a bottom surface of a base model as a plane of projection.

In an embodiment, the data processing device 120 may design a die by forming a plurality of partial layers between the boundary line 1013 and the target plane 1015. In order to form the plurality of partial layers, the data processing device 120 may obtain a length of a curved surface surrounded by the boundary line 1013, that is, an average side of a mesh included in the target curved surface 1010. In an embodiment, the data processing device 120 may obtain a distance between a point farthest from the target plane 1015 and a point closest from the target plane 1015 among points included in the boundary line 1013.

For example, in FIGS. 10A and 10B, when the point of the boundary line 1013 closest to the target plane 1015 is P₁ and the point of the boundary line 1013 closest to the target plane 1015 is P₂, the data processing device 120 may obtain a distance between the point P₁ and the point P₂ as the maximum distance.

In an embodiment, the data processing device 120 may obtain the number N of partial layers (N is a natural number) by dividing the maximum distance between the point P₁ and the point P₂ by the length of the average side of meshes. The partial layer is a layer forming a die and may be a segment.

In an embodiment, the data processing device 120 may design a die by forming the N partial layers each having the length of the average side of meshes at the maximum distance between the point P₁ and the point P₂.

In an embodiment, the data processing device 120 may form the N partial layers at once. For example, referring to FIG. 10A, the data processing device 120 may simultaneously form the N partial layers each having the length of the average side of meshes between the point P₁ of the boundary line 1013 farthest from the target plane 1015 and the point P₂ of the boundary line 1013 closest from the target plane 1015.

When a plane parallel to the target plane 1015 while passing through the point P₂ of the boundary line 1013 closest to the target plane 1015 is referred to as a temporary plane 1016, the data processing device 120 may generate a side surface of the die surrounded by a projection line by projecting the projection line in a direction of the temporary plane 1016 from each point of the boundary line 1013. In an embodiment, the data processing device 120 may form the side surface of the die into the N partial layers.

As described above, when designing a die of the object 1010, the data processing device 120 may determine whether there is an interference region between the boundary line 1013 and a plane of projection by using a projection diagram drawn by the projection line on the plane of projection. In an embodiment, when two or more single closed curves are included in the projection diagram, the data processing device 120 may design a die of the object 1010 with a mold surrounded by a projection line only from points on a boundary line corresponding to a single closed curve having the longest length among the two or more single closed curves. In this case, there may be empty space between the interference region and a point of a boundary line corresponding to the remaining single closed curve rather than the single closed curve having the longest length.

For example, in FIG. 10A, a region denoted by the reference numeral 1014 indicates a position of the boundary line corresponding to the remaining single closed curve among the two or more single closed curves.

In an embodiment, the data processing device 120 may fill the empty space between the interference region and a point of a boundary line corresponding to the remaining single closed curve by using a mesh connecting points around the empty space. For example, the data processing device 120 may identify points on a projection line at the shortest distance from a point of the boundary line corresponding to the remaining single closed curve excluding the longest single closed curve. The data processing device 120 may generate a mesh by connecting the point of the boundary line corresponding to the remaining single closed curve to the points on the projection line at the shortest distance from the boundary line corresponding to the remaining single closed curve.

In an embodiment, the data processing device 120 may fill an empty space between the interference region and the point of the boundary line corresponding to the remaining single closed curves with a mesh.

In this case, as in the region denoted by reference numeral 1014 in FIG. 10A, an empty space 1014 generated due to the interference region is naturally filled with a mesh to which surrounding points are connected.

In an embodiment, the data processing device 120 may form the N partial layers between the boundary line 1013 and the temporary plane 1016, and then form partial layers between the temporary plane 1016 and the target plane 1015. In an embodiment, the data processing device 120 may obtain the number M of partial layers by obtaining a distance between the temporary plane 1016 and the target plane 1015, and dividing the distance by the length of the average side of meshes. The data processing device 120 may design the die by filling between the temporary plane 1016 and the target plane 1015 with the M partial layers each having the length of the average side of meshes. FIG. 10B shows the data processing device 120 designing a die by filling between the temporary plane 1016 and the target plane 1015 with the M partial layers.

In an embodiment, the data processing device 120 may design the die in partial layers and then smoothly process a surface of the die. In an embodiment, the data processing device 120 may remove irregularly formed dots or lines, or smooth the surface of the die by using a filter, etc. to make the surface of the die smooth without angular parts.

FIG. 10B illustrates a case where the data processing device 120 designs the die and then smoothly processes the surface of the die. Upon comparing FIGS. 10A and 10B, it may be seen that upper and side surfaces of the die shown in FIG. 10B are formed in a smooth and natural curved surface, unlike the die shown in FIG. 10A.

As described above, by forming the die in the partial layer corresponding to the length of the average side of meshes, the data processing device 120 may form the curved surface above the boundary line 1013 and the partial layer below the boundary line 1013 in meshes of the same size.

In addition, the data processing device 120 may form a plurality of partial layers below the boundary line 1013 at once.

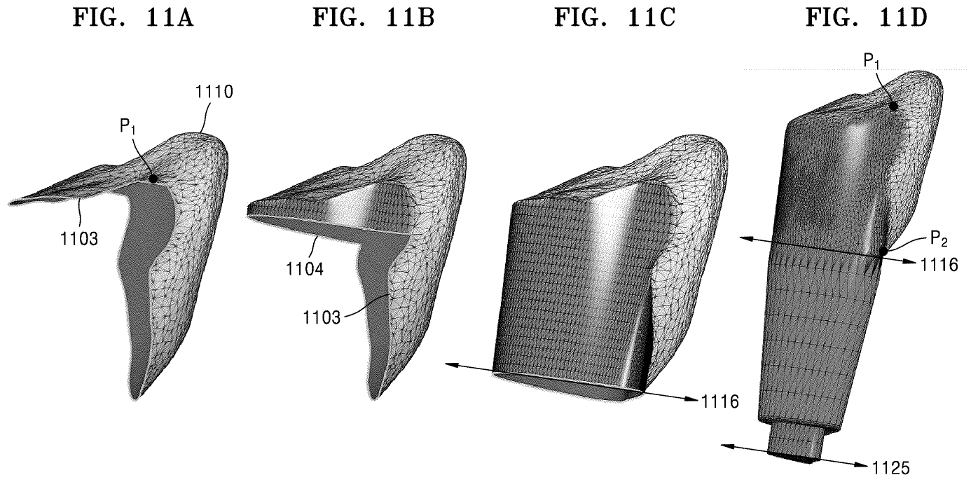

FIGS. 11A, 11B, 11C, and 11D are diagrams illustrating the data processing device 120 sequentially forming partial layers when forming a die of an object, according to an embodiment.

A target curved surface 1110 shown in FIGS. 11A, 11B, 11C, and 11D may be the same as the target curved surface shown in FIG. 6 and the target curved surface 1010 shown in FIG. 10.

In an embodiment, the data processing device 120 may obtain a length of an average side of a mesh included in a curved surface surrounded by a boundary line 1103.

In an embodiment, the data processing device 120 may obtain the number N of partial layers by obtaining a distance between the point P₁ of a boundary line farthest from a target plane 1125 and the point P₂ of the boundary line closest from the target plane 1125, and dividing the distance by the length of the average side of meshes.

In an embodiment, the data processing device 120 may design a die by sequentially forming the N partial layers each having the length of the average side of meshes between the point P₁ and the point P₂.

FIGS. 11A to 11C illustrate the data processing device 120 sequentially forming the N partial layers between the point P₁ and the point P₂.

FIG. 11A illustrates the data processing device 120 forming a first partial layer. In an embodiment, the data processing device 120 may form the first partial layer below the point P₁ of a boundary line farthest from the target plane 1125. The first partial layer may have the length of the average side of meshes.

In an embodiment, the data processing device 120 may obtain a new boundary line by connecting a line of the first partial layer to a boundary line below which the first partial layer is not formed. In an embodiment, the data processing device 120 may form a second partial layer below the first partial layer by using a projection line projected from the new boundary line toward the target plane 1125 and having the length of the average side of meshes.

In an embodiment, the data processing device 120 may form a k layer, and then sequentially form a k+1 partial layer below the k layer by using the same method. For example, referring to FIG. 11B, reference numeral 1104 may denote a lower line of the k layer, and reference numeral 1103 may denote a boundary line in which the k layer is not formed. The data processing device 120 may identify a curve connecting a lower line 1104 of the k layer to a boundary line 1103 in which the k layer is not formed as a new boundary line. The data processing device 120 may form the k+1 layer below the k layer with a projection line projected from the new boundary line toward the target plane 1125 and having the length of the average side of meshes.

As described above, in an embodiment, when the data processing device 120 sequentially forms a plurality of partial layers between a boundary line and a target plane without simultaneously forming the plurality of partial layers, boundary lines continue to change. When boundary lines change, an interference region may or may not be included between a boundary line and a plane of projection. For example, there is no interference region between a new boundary line generated by forming the k partial layer and the target plane, but there may be an interference region between a new boundary line generated by forming the k+1 partial layer and the target plane.

In an embodiment, whenever a new boundary line is obtained, the data processing device 120 may identify whether an interference region is included between the new boundary line and the target plane 1125. That is, in an embodiment, whenever a new boundary line is obtained, the data processing device 120 may identify whether two or more single closed curves are included in a projection diagram formed by a projection line projected from the new boundary line on the target plane 1125.

In an embodiment, when two or more single closed curves are included in the projection diagram, the data processing device 120 may identify the longest single closed curve among the two or more single closed curves, and may identify points of a new boundary line corresponding to the longest single closed curve. The points of the new boundary line corresponding to the longest single pebble may mean points on the new boundary line to which the longest single closed curve and the projection line are connected.

In an embodiment, the data processing device 120 may form a partial layer by using a projection line projected from only points of a new boundary line.

In an embodiment, the data processing device 120 may design a die by filling the distance between the point P₁ and the point P₂, that is, a distance between the point P₁ and a temporary plane 1116 with the N partial layers.

In an embodiment, as shown in FIG. 11D, the data processing device 120 may design a die by obtaining the number M of partial layers by dividing the distance between the temporary plane 1116 and the target plane 1125 by the length of the average side of meshes included in the curved surface surrounding the boundary line 1103, and filling between the temporary plane 1116 and the target plane 1125 with M partial layers.

As described above, according to an embodiment, the data processing device 120 may design the die of the object by sequentially filling between the boundary line 1103 and the target plane 1125 with partial layers each having the length of the average side of meshes.

In addition, according to an embodiment, the data processing device 120 may obtain a new boundary line whenever sequentially forming a plurality of partial layers are, and determine whether there is an interference region between the new boundary line and the target plane 1125.

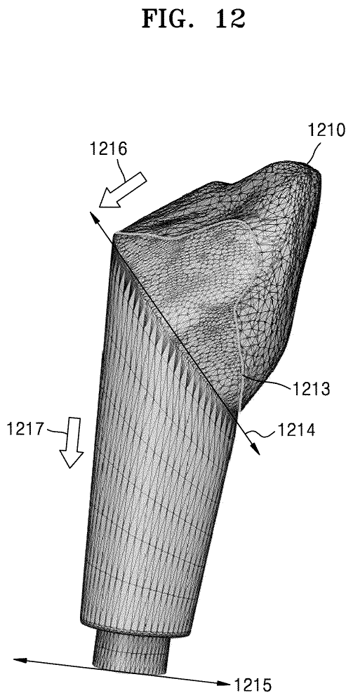

FIG. 12 is a diagram illustrating the data processing device 120 designing a die of an object, according to an embodiment.

A target curved surface 1210 shown in FIG. 12 may be the same as the target curved surfaces 1010 and 1110 shown in FIGS. 6, 10, and 11.

Referring to FIG. 12, the data processing device 120 may obtain 3D scan data of an object and identify a boundary line 1213 selected for generation of a die from 3D scan data of the object.

In an embodiment, the data processing device 120 may not design a die by connecting the boundary line 1213 to a target plane 1215 as described in FIG. 10 or 11, but may design a die by generating a first plane of projection 1214 between the boundary line 1213 and the target plane 1215 and connecting the boundary line 1213 to the first plane of projection 1214 and the first plane of projection 1214 to the target plane 1215.

A projection diagram projected from a boundary line to a plane of projection has various forms depending on a projection angle. That is, depending on a projection direction, whether an interference region is included between the boundary line and the plane of projection, the degree of inclusion of the interference region, the position of inclusion of the interference region, etc. are different.

In an embodiment, the data processing device 120 may obtain a convex hull curved surface of the boundary line by connecting points included in the boundary line 1213.

The convex hull curved surface may be a closed curved surface generated by connecting the points included in the boundary line 1213. The convex hull curved surface may be the minimum block shell including all meshes generated by the points included in the boundary line 1213.

In an embodiment, the data processing device 120 may obtain a normal vector of a convex hull curved surface of a boundary line.

In an embodiment, the data processing device 120 may obtain a final normal vector direction by obtaining normal vectors of a mesh forming a convex hull curved surface, and obtaining an average of the normal vectors of the mesh. For example, in FIG. 12, the normal vector direction of the convex hull curved surface of the boundary line may be a direction parallel to reference numeral 1216 of FIG. 12.

In an embodiment, the data processing device 120 may generate a plane perpendicular to the normal vector of the convex hull curved surface as a first plane of projection 1214.

In an embodiment, the data processing device 120 may determine the normal vector direction in consideration of a weight according to the area of the mesh. For example, the data processing device 120 may obtain a weighted normal vector by assigning a larger weight to the normal vector of the mesh as the area of the mesh increases, and a smaller weight of the normal vector of the mesh as the area of the mesh decreases. The data processing device 120 may obtain the weighted normal vector by internalizing the normal vector in the area of the mesh and averaging the internalized normal vector for all meshes.

In an embodiment, as shown in FIG. 12, the data processing device 120 may obtain a plane separated by the shortest distance from the boundary line 1213, among planes having a slope perpendicular to the normal vector direction, as the first plane of projection 1214. That is, the data processing device 120 may obtain a plane having a slope perpendicular to the normal vector direction and having the shortest separation distance from the boundary line 1213 or contacting the boundary line 1213 as the first plane of projection 1214.

As described above, when the plane perpendicular to the normal vector of the convex hull curved surface of the boundary line is used as the first plane of projection 1214, the probability that a projection line projected from the boundary line 1213 to the target plane 1215 meets an interference region may be lower than the probability that a projection line projected from the boundary line 1213 to the first plane of projection 1214 meets the interference region.

Referring to FIG. 12, the first plane of projection 1214 may be disposed below the boundary line 1213. The first plane of projection 1214 may be disposed between the boundary line 1213 and the target plane 1215 to have the slope perpendicular to the normal vector direction.

In an embodiment, the data processing device 120 may obtain a first projection line projected from a point of the boundary line 1213 and meeting the first plane of projection 1214. In FIG. 12, a projection direction of the first projection line may be denoted by, for example, reference numeral 1216. The first projection line may be a projection line that vertically meets the first plane of projection 1214.

In an embodiment, the data processing device 120 may obtain a second projection line that is projected from a point where the first projection line and the first plane of projection 1214 meet and meets a second plane of projection, by using the target plane 1215 as the second plane of projection. In FIG. 12, a projection direction of the second projection line may be denoted by, for example, reference numeral 1217.

In an embodiment, the data processing device 120 may design a die by using a curved surface surrounded by the boundary line 1213, that is, a mold surrounded by a target curved surface 1210 and the first projection line and the second projection line.

In this way, instead of generating a die by directly connecting the boundary line 1213 to the target plane 1215, the data processing device 120 may generate a die by firstly connecting the boundary line 1213 to the first plane of projection 1214 and secondly connecting the first plane of projection 1214 and the second plane of projection 1215. The data processing device 120 may design a die by firstly connecting the boundary line 1213 to the first plane of projection 1214, thereby reducing the probability that an interference region is included between the boundary line 1213 and the first plane of projection 1214.

In some cases, an interference region may also be included between the boundary line 1213 and the first plane of projection 1214. In an embodiment, the data processing device 120 may determine whether there is an interference region between the boundary line 1213 and the first plane of projection 1214 by identifying whether two or more single closed curves are included in a first projection diagram formed by the first projection line on the first plane of projection 1214.

In an embodiment, when it is determined that the interference region is included, the data processing device 120 may generate a die by using a projection line projected from only points of the boundary line corresponding to the longest single closed curve among the two or more single closed curves as the first projection line.

In an embodiment, the data processing device 120 may generate a triangle, that is, a mesh, by connecting a point of a boundary line corresponding to the remaining single closed curve excluding the longest single closed curve to a point on a projection line at the shortest distance from the point of the boundary line corresponding to the remaining single closed curve, and fill an empty space between the boundary line corresponding to the remaining single closed curve and the interference region with a mesh.

In an embodiment, the data processing device 120 may obtain the number N of partial layers by obtaining a length of an average side of meshes included in the curved surface formed by the boundary line 1213, and dividing a distance between a point of the boundary line farthest from the first plane of projection 1214 and a point of the boundary line closest to the first plane of projection 1214 by the length of the average side of meshes. When the first plane of projection 1214 contacts the boundary line 1213, a distance between the first plane of projection 1214 and the point of the boundary line closest to the first plane of projection 1214 is 0.

In an embodiment, the data processing device 120 may design a die by forming N partial layers each having the length of the average side of meshes between the point of the boundary line farthest from the first plane of projection 1214 and the first plane of projection 1214.

In an embodiment, the data processing device 120 may design a die by forming the N partial layers at once between the boundary line 1213 and the first plane of projection 1214.

Alternatively, in an embodiment, the data processing device 120 may sequentially form the N partial layers. For example, the data processing device 120 may form a first partial layer below the point of the boundary line farthest from the first plane of projection 1214 with a projection line projected from the point of the boundary line farthest from the first plane of projection 1214 toward the first plane of projection 1214, i.e., in a direction parallel to a normal vector of a convex hull surface of the boundary line and having the length of the average side of meshes.

In an embodiment, the data processing device 120 may obtain a new boundary line by connecting a line of the first partial layer to a boundary line below which the first partial layer is not formed, and design a die by sequentially forming a second partial layer below the first partial layer, with a projection line projected from a point of the new boundary line toward the first plane of projection 1214 and having the length of the average side of meshes.

In an embodiment, whenever a new boundary line is obtained, the data processing device 120 may determine whether there is an interference region between the new boundary line and the first plane of projection 1214. In an embodiment, the data processing device 120 may form the second partial layer by using a projection line projected only from points of a new boundary line corresponding to the longest single closed curve among two or more single closed curves based on the fact that the two or more single closed curves are included in a projection diagram formed by the projection line projected from the point of the new boundary line on the first plane of projection 1214.

In an embodiment, the data processing device 120 may obtain the number M of partial layers by dividing the maximum distance among vertical distances between the first plane of projection 1214 and the second plane of projection by the length of the average side of meshes, by using the target plane 1215 as the second plane of projection.

In an embodiment, the data processing device 120 may form M or fewer partial layers each having the length of the average side of meshes between the first plane of projection 1214 and the second plane of projection 1215.

In an embodiment, the data processing device 120 may perform surface processing such as smoothing on a partial layer such that the partial layer and the object 1210 are naturally connected to each other.

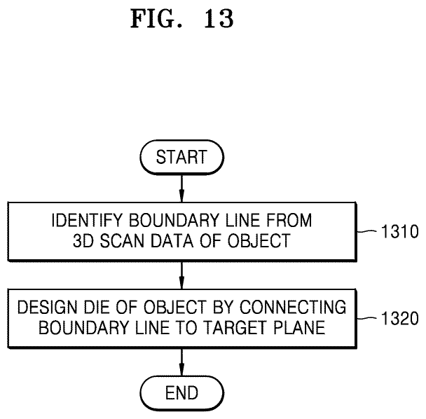

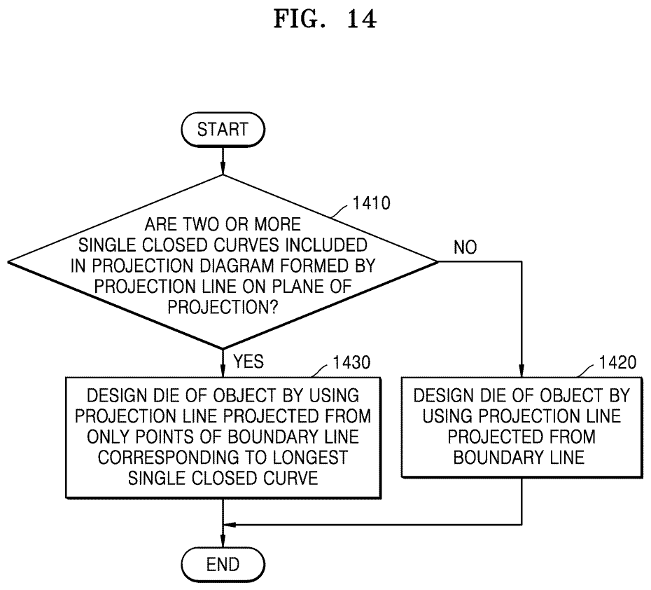

FIG. 13 is a flowchart illustrating a data processing method according to an embodiment.

In an embodiment, the data processing device 120 may obtain 3D scan data of an object for which a die is to be generated.

In an embodiment, the data processing device 120 may identify the object from the 3D scan data of the object.

In an embodiment, the data processing device 120 may identify a boundary line from the 3D scan data of the object (operation 1310). The data processing device 120 may receive a boundary line from a user or automatically generate a boundary line for an object selected by the user.

In an embodiment, the data processing device 120 may identify a target plane in a base model. The data processing device 120 may receive a selection of a target plane from the user or may automatically identify the target plane.

In an embodiment, the data processing device 120 may design a die of the object by connecting the boundary line to the target plane (operation 1320).

FIG. 14 is a flowchart illustrating a data processing method according to an embodiment.

In an embodiment, the data processing device 120 may obtain a plane of projection where a projection line projected from a boundary line meets. In an embodiment, the plane of projection may be a target plane or a first plane of projection located between the boundary line and the target plane.

In an embodiment, the data processing device 120 may determine whether there is an interference region between the boundary line and the plane of projection.

In an embodiment, the data processing device 120 may determine whether two or more single closed curves are included in a projection diagram formed by a projection line on the plane of projection (operation 1410).

In an embodiment, when it is determined that the two or more single closed curves are not included in the projection diagram, the data processing device 120 may design a die of the object by using the projection line projected from the boundary line (operation 1420).

In an embodiment, when it is determined that the two or more single closed curves are included in the projection diagram, the data processing device 120 may design the die of the object by using a projection line projected from only points of the boundary line corresponding to the longest single closed curve (operation 1430).

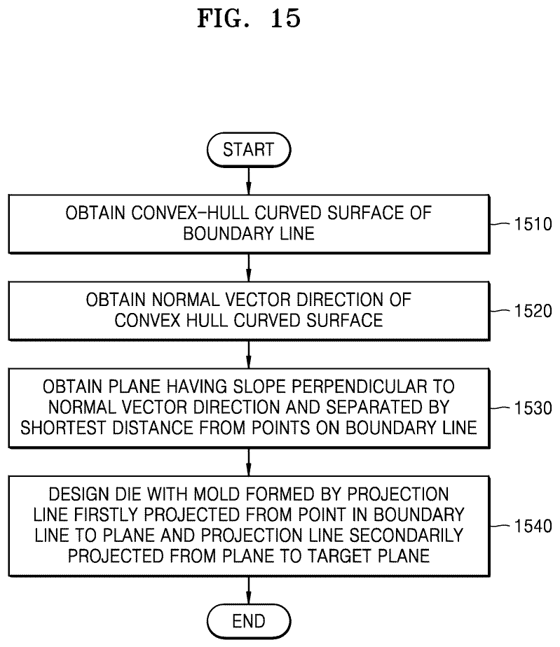

FIG. 15 is a flowchart illustrating a data processing method according to an embodiment.

In an embodiment, the data processing device 120 may obtain a convex-hull curved surface of a boundary line (operation 1510).

In an embodiment, the data processing device 120 may obtain a normal vector direction of the convex hull curved surface (operation 1520). The normal vector direction of the convex hull curved surface may be a weighted normal vector direction in which a weight according to the area of a mesh forming the convex hull curved surface is considered.

In an embodiment, the data processing device 120 may obtain a plane having a slope perpendicular to the normal vector direction and separated by the shortest distance from the points on the boundary line (operation 1530).

In an embodiment, the data processing device 120 may design a die with a mold formed by a projection line firstly projected from the point in the boundary line to a plane and a projection line secondarily projected from the plane to the target plane (operation 1540).

A data processing method according to an embodiment of the present disclosure may be recorded on a computer-readable recording medium by being implemented in a form of program commands executed by using various computers. Also, an embodiment of the present disclosure may include a computer-readable storage medium having recorded thereon at least one program including at least one instruction for executing the data processing method.

The data processing method according to an embodiment of the present disclosure may be implemented by a computer program product including a computer-readable recording medium having recorded thereon a program for executing the data processing method including identifying a boundary line selected for generation of a die in 3D scan data of an object, and designing the die of the object by connecting the boundary line to a target plane included in a bottom surface of a base model.

The computer-readable recording medium may include at least one of a program command, a data file, or a data structure. Examples of the computer-readable recording medium include magnetic media such as hard disks, floppy disks, and magnetic tapes, optical media such as CD-ROMs and DVDs, magneto-optical media such as floptical disks, and hardware devices configured to store and perform program commands, such as read-only memory (ROM), random-access memory (RAM), and flash memory.

A machine-readable storage medium may be provided in the form of a non-transitory storage medium. The “non-transitory storage medium” may denote that a storage medium is a tangible device. The “non-transitory storage medium” may include a buffer where data is temporarily stored.

According to an embodiment, a data processing method according to various embodiments in the present specification may be provided by being included in a computer program product. The computer program product may be distributed in the form of the machine-readable storage medium (e.g., a compact disc read-only memory (CD-ROM). Alternatively, the computer program product may be distributed (e.g., downloaded or uploaded) directly or online through an application store (e.g., PlayStore™) or between two user devices (e.g., smartphones). In detail, the computer program product according to an embodiment may include a storage medium having recorded thereon a program including at least one instruction for executing the data processing method according to an embodiment.

While the embodiments have been particularly shown and described in detail, it will be understood by one of ordinary skill in the art that various changes in form and details may be made therein without departing from the spirit and scope of the present disclosure as defined by the following claims.

1. A data processing method performed by a data processing device, the data processing method comprising:
identifying a boundary line selected for generation of a die, from three-dimensional (3D) scan data of an object; and
designing a die of the object by connecting the boundary line and a target plane included in a bottom surface of a base model.

2. The data processing method of claim 1, wherein the designing of the die of the object includes:
identifying a projection direction;
identifying, as the target plane, a region where a projection line projected in the projection direction from a point of the boundary line meets the bottom surface of the base model; and
designing the die of the object by using a mold surrounded by the projecting line.

3. The data processing method of claim 2, further comprising: displaying the designed die,
wherein a cross-section of the displayed die includes one single closed curve.

4. The data processing method of claim 3, wherein the designing of the die of the object includes, based on a fact that two or more single closed curves are included in a projection diagram formed by the projection line on a plane of projection, designing the die of the object by using a projection line projected from only points of a boundary line corresponding to a longest single closed curve among the two or more single closed curves.

5. The data processing method of claim 4, wherein the designing of the die of the object further includes
generating a mesh by connecting a point of a boundary line corresponding to a remaining single closed curve excluding the longest single closed curve to a point on a projection line at a shortest distance from the point of the boundary line corresponding to the remaining single closed curve; and
filling, with the mesh, empty space below the boundary line corresponding to the remaining single closed curve.

6. The data processing method of claim 3, wherein the designing of the die of the object includes
obtaining a length of an average side of meshes included in a curved surface surrounded by the boundary line;
obtaining a number N of partial layers (N is a natural number) by dividing a distance between a point of the boundary line farthest from the target plane and a point of the boundary line closest to the target plane by the length of the average side of meshes; and
designing the die by forming the N partial layers, each having the length of the average side of meshes, between the farthest point of the boundary line and the closest point of the boundary line.

7. The data processing method of claim 6, wherein the designing of the die by forming the N partial layers includes
forming a first partial layer below the farthest point of the boundary line with a projection line projected from the farthest point of the boundary line toward the target plane and having the length of the average side of meshes;
obtaining a new boundary line by connecting a line of the first partial layer to a boundary line below which the first partial layer is not formed; and
forming a second partial layer below the first partial layer with a projection line projected from a point of the new boundary line toward the target plane and having the length of the average side of meshes.

8. The data processing method of claim 7, wherein the forming of the second partial layer includes, based on a fact that two or more single closed curves are included in a projection diagram formed on the target plane by the projection line projected from the point of the new boundary line, forming the second partial layer by using a projection line projected from only points of the new boundary line corresponding to a longest single closed curve among the two or more single closed curves.

9. The data processing method of claim 1, wherein the designing of the die of the object includes
obtaining, as a first plane of projection, a plane that has a slope perpendicular to a normal vector of a closed curve surface generated using points included in the boundary line and is separated by a shortest distance from the boundary line;
obtaining a first projection line projected from a point of the boundary line and vertically meeting the first plane of projection;
obtaining a second projection line that is projected from a point where the first projection line and the first plane of projection meet and meets a second plane of projection, by using the target plane as the second plane of projection; and
designing the die by using a mold surrounded by the first projection line and the second projection line.

10. The data processing method of claim 9, further comprising: displaying the designed die,
wherein a cross-section of the displayed die includes one single closed curve.

11. The data processing method of claim 10, wherein the obtaining of the first projection line includes,
based on a fact that two or more single closed curves are included in a projection diagram formed by the first projection line on the first plane of projection, obtaining, as the first projection line, a projection line projected from only points of the boundary line corresponding to a longest single closed curve among the two or more single closed curves.

12. The data processing method of claim 11, wherein the designing of the die of the object further includes
generating a mesh by connecting a point of a boundary line corresponding to a remaining single closed curve excluding the longest single closed curve to a point on a projection line at a shortest distance from the point of the boundary line corresponding to the remaining single closed curve; and
filling, with the mesh, empty space below the boundary line corresponding to the remaining single closed curve.

13. The data processing method of claim 9, wherein the obtaining of, as the first plane of projection, the plane includes:
obtaining the closed curved surface generated using the points included in the boundary line;
obtaining a direction of the normal vector of the closed curved surface; and
obtaining, as the first plane of projection, the plane that has the slope perpendicular to the normal vector and is separated by the shortest distance from the boundary line, among planes in contact with or spaced apart from the boundary line.

14. The data processing method of claim 13, wherein the direction of the normal vector is a direction of a weighted normal vector obtained by adding a weight according to an area of a mesh forming the closed curved surface.

15. The data processing method of claim 10, wherein the designing of the die of the object includes
obtaining a number N of partial layers (N is a natural number) by dividing a distance between a farthest point of the boundary line from the first plane of projection and the first plane of projection by the length of the average side of meshes; and
forming the N partial layers each having the length of the average side of meshes, between the farthest point of the boundary line and the first plane of projection.

16. The data processing method of claim 15, wherein the forming of the N partial layers includes
forming a first partial layer below the farthest point of the boundary line with a projection line projected from the farthest point of the boundary line toward the first plane of projection and having the length of the average side of meshes;
obtaining a new boundary line by connecting a line of the first partial layer and a boundary line below which the first partial layer is not formed; and
forming a second partial layer below the first partial layer with a projection line projected from a point of the new boundary line toward the first plane of projection and having the length of the average side of meshes.

17. The data processing method of claim 16, wherein the forming of the second partial layer includes,
based on a fact that two or more single closed curves are included in a projection diagram formed on the first plane of projection by the projection line projected from the point of the new boundary line, forming the second partial layer by using a projection line projected from only points of the new boundary line corresponding to a longest single closed curve among the two or more single closed curves.

18. The data processing method of claim 15, wherein the designing of the die of the object further includes
obtaining a number M of partial layers (M is a natural number) by dividing a maximum distance between the first plane of projection and a second plane of projection by the length of the average side of meshes, by using the target plane as the second plane of projection; and
forming M or fewer partial layers each having the length of the average side of meshes, between the first plane of projection and the second plane of projection.

19. A data processing device comprising:
one or more processors configured to execute one or more instructions, wherein the one or more processors are configured to execute the one or more instructions to:
identify a boundary line selected for generation of a die, from three-dimensional (3D) scan data of an object; and
design a die of the object by connecting the boundary line and a target plane included in a bottom surface of a base model.

20. A computer-readable recording medium having recorded thereon a program for implementing a data processing method comprising:
identifying a boundary line selected for generation of a die, from three-dimensional (3D) scan data of an object; and
designing a die of the object by connecting the boundary line and a target plane included in a bottom surface of a base model.
